(19) United States
(12) Patent Application Publication
(10) Pub. No.: US 2008/0161865 A1
(43) Pub. Date: Jul. 3, 2008

(54) IMPLANTABLE VESSEL STIMULATION DEVICE COATING

(75) Inventor: Jeffrey J. Hagen, Plymouth, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Maple Grove, MN (US)

(21) Appl. No.: 11/617,098

(22) Filed: Dec. 28, 2006

(51) Int. Cl. A61N 1/05

(52) U.S. Cl. 607/2

(57) ABSTRACT

A device positionable on a vascular structure for stimulating the vascular structure to elicit a physiologic response. The device includes a base having generally opposed inner and outer surfaces and a hydrophilic material presented on at least a portion of the inner surface presenting a lubricious surface for selectively positioning the device on the vascular structure. The device further includes an electrode structure presented with the base to provide stimulation to the vascular structure, wherein the base and electrodes are configured to conform to at least a portion of the vascular structure to maintain an intimate vascular structure-electrode interface.

### BACKGROUND OF THE INVENTION

1. Field of the Invention

The present invention generally relates to implantable devices for treating and managing cardiovascular and renal disorders. More particularly, the present invention relates to hydrophilic coatings for implantable vessel stimulation devices enabling efficient device placement and effective vessel-device interfaces while facilitating the inhibition of inflammatory responses and thrombus and fibrosis formation at implantation sites.

Hypertension (high blood pressure) is a major cardiovascular disease estimated to affect millions people annually in the United Sates alone. Hypertension occurs when the body's smaller blood vessels constrict causing an increase in blood pressure. Because the blood vessels constrict, the heart generally must work harder to maintain blood flow at the higher pressures. Although the body can tolerate shorter periods of increased blood pressure, sustained hypertension can eventually result in damage to the kidneys, brain, eyes, and other tissues. The elevated blood pressure can also damage the lining of the blood vessels, accelerating atherosclerosis and increasing the likelihood of a blood clot forming that can lead to a heart attack and/or stroke. Sustained high blood pressure can also result in an enlarged and damaged heart that can lead to heart failure. Heart failure is the final common expression of a variety of cardiovascular disorders, including ischemic heart disease, and is characterized by an inability of the heart to pump enough blood to meet the body's needs.

Heart failure often results in the activation of a number of body systems to compensate for the heart's inability to pump sufficient blood. Many of these responses are mediated by an increase in the level of activation of the sympathetic nervous system, in addition to the activation of multiple other neurohormonal responses. Generally, this sympathetic nervous system activation signals the heart to increase heart rate and force of contraction to increase the cardiac output. It signals the kidneys to expand the blood volume by retaining sodium and water. It also signals the arterioles to constrict to elevate the blood pressure. The cardiac, renal, and vascular responses increase the workload of the heart, further accelerating myocardial damage and exacerbating heart failure.

The wall of the carotid sinus, a structure at the bifurcation of the common carotid arteries, contains stretch receptors (baroreceptors) sensitive to the blood pressure. These receptors send signals via the carotid sinus nerve to the brain, which in turn regulates the cardiovascular system to maintain normal blood pressure (the baroreflex), in part through control of the sympathetic nervous system.

Electrical stimulation of the carotid sinus (baropacing) has been used to treat high blood pressure and angina by reducing blood pressure and the workload of the heart. For example, U.S. Pat. No. 6,073,048 to Kieval et al. discloses systems and devices for activating baroreceptors, indicating an increase in blood pressure and signaling the brain to reduce the body's blood pressure and level of sympathetic nervous system and neurohormonal activation and increase parasympathetic nervous system activation.

Baroreceptor activation devices can be positioned either within the carotid artery (intravascular) or external to the carotid arteries (extravascular). When intravascular, the activation devices can be placed inside a vessel, such as near the baroreceptors at the carotid sinus where the carotid artery bifurcates into an external carotid artery and an internal carotid artery. Care generally must be taken when placing any device intravascularly, as interaction between the device and vascular lumen can present potential for damage to the device and or the inner walls of the vascular lumen.

When using extravascular baroreceptor activation devices, the devices can be placed on or about an exterior portion of a vessel and selectively positioned near the baroreceptors, such as at the carotid sinus where the carotid artery bifurcates into an external carotid artery and an internal carotid artery. As with intravascular activation devices, care generally must be taken when placing an extravascular activation device near the baroreceptors at the carotid sinus, as any friction between the device and vascular wall can present potential for damage to the device and or the outer wall of a vascular lumen, and may cause constrictions or turbulence within the vessel.

Moreover, the functionality of the device can depend upon the inner surface of the device being effectively coupled and in good contact with the exterior vascular surface, such that effective mechanical, electrical, thermal, chemical, biological, or other activation of the wall or structure in the wall can occur. However, when extravascular devices are implanted in the body and placed or about a vascular structure, an immune response can cause an inflammatory response followed by encapsulation of the internal surface of the device with tissue. When this type of response occurs, the mechanical, electrical, thermal, chemical, or biological characteristics of the vessel-electrode interface can degrade causing the device to fail or function ineffectively.

Further, tissue building up on the exterior of the device can potentially contract the device on the artery, for example, can cause a false parameter indicative of the need to modify the baroreflex system activity. For example, in a baroreceptor activation device, this can then lead the control system to generate a control signal activating the baroreceptor activation device to induce a baroreceptor signal that is perceived by the brain to be apparent excessive blood pressure.

Therefore, it would be desirable to produce an improved implantable vessel stimulation device overcoming deficiencies with existing designs.

2. Description of the Background Art

Certain types of implantable baroreceptor activation devices are designed to be placed over an artery or vessel (extravascular). For example, particular implantable conductive baroreceptor activation device structures (e.g., electrodes) can be wrapped around a carotid sinus or other vascular structure. Examples of such electrodes are disclosed in U.S. Patent Publication No. 2003/0060857, U.S. Patent Publication No. 2004/0010303, U.S. Patent Publication No. 2006/0004430, and U.S. Patent Publication No. 2006/0111626. The electrode structures can be held in place on or about the carotid artery, for example, proximate a baroreceptor to enable baroreceptor stimulation to induce the baroreflex to control hypertension or other conditions.

### BRIEF SUMMARY OF THE INVENTION

The vessel stimulation methods according to the various embodiments of the present invention generally include directing stimulation to a vessel wall for the purposes of eliciting a physiologic response. The method can include providing a device having a base structure having a hydrophilic material presented therewith and an electrode structure thereon. The method further can include selectively positioning the device on a vessel wall, extending the base structure around at least a portion thereof, and activating, deactivating, or otherwise modulating the device to provide stimulation to the vessel wall with the electrode for the purposes of eliciting a physiologic response. The method can also include determining an effective position for providing stimulation to the vessel wall before or during selectively positioning the device on the vessel wall. The determination step can include applying an electrical stimulus and observing a response.

In various embodiments as described herein, the step of providing stimulation to the vessel wall can be for purposes of eliciting a baroreflex response. Specifically, the vessel wall can include one or more baroreceptors therein, wherein the step of selectively positioning the device on the vessel wall comprises determining the location of the one or more baroreceptors and effecting movement of the device such that the device is proximate the one or more baroreceptors. The step of activating, deactivating, or otherwise modulating the device can be used to provide stimulation to the vascular structure for purposes of eliciting the baroreflex response by activating a baroreceptor or nerves emanating therefrom.

The baroreceptor active device according to various embodiments of the present invention is selectively positionable on a vascular structure for providing stimulation to the vascular structure for purposes of eliciting a baroreflex response. The device includes a base structure having inner and outer surfaces, typically on opposite sides of the base structure, and a hydrophilic material presented on at least a portion of the inner surface presenting a lubricious surface for selectively positioning the device on the vascular structure. The device further includes an electrode structure presented with the base structure operable to provide stimulation to the vascular structure, wherein the base structure and electrodes are configured to conform to at least a portion of the vascular structure to maintain an intimate vascular structure-electrode interface.

In one embodiment, an anti-inflammatory agent can be presented with the hydrophilic material. In some embodiments, the base structure can include electrical insulation, such that the stimulation is directionally provided toward the vascular structure. In another embodiment, the base structure can further include hydrophilic material disposed on the outer surface of the base structure. In another embodiment, the base structure can further include a hydrophobic material presented intermediate the hydrophilic material and the inner surface. In yet another embodiment, the base structure can further include a hydrophobic material presented intermediate the hydrophilic material and the inner surface and hydrophilic material disposed on the outer surface of the base structure. In one embodiment, the vascular structure includes a portion of a carotid artery and the base structure has a length enabling the base structure to extend substantially around the portion of the carotid artery conforming thereto.

The method of providing stimulation to the vascular structure for purposes of eliciting a baroreflex response according to the various embodiments includes providing an extravascular device comprising a base structure having a hydrophilic material presented therewith and an electrode structure thereon, selectively positioning the device on the vascular structure, extending the base structure around at least a portion of the vascular structure and optionally operably coupling the base structure thereto and selectively positioning and re-positioning the device on the vascular structure. The hydrophilic material can provide a lubricious surface for effecting movement of the device with respect to the vascular structure during positioning and re-positioning, and activating, deactivating, or otherwise modulating the device to provide stimulation to the vascular structure.

In an embodiment, the method can also include determining an effective position for providing stimulation to the vascular structure before or during selectively positioning the device on the vascular structure. The step of determining an effective position can include applying an electrical stimulus and observing a response.

In one embodiment, the device can include a belt mechanism comprising a strap and a buckle, wherein the step of operably coupling the base structure to the vascular structure comprising engaging the strap with the buckle such that the buckle retains at least a portion of the strap. The step of operably coupling the base structure to the vascular structure can also include suturing the base structure to the vascular structure. The base structure can include a hydrophobic material presented therewith enabling adhesion between the vascular structure and hydrophobic material.

The vascular structure can be a carotid artery having baroreceptors therein, wherein the step of selectively positioning the device on the vascular structure includes determining the location of one or more baroreceptors by, for example, measuring the efficacy of a stimulation and effecting movement of the device such that the device is more optimally positioned relative to one or more baroreceptors.

A method of selectively positioning a device on a vascular structure for providing stimulation to the vascular structure for purposes of eliciting a physiologic response according to the various embodiments can include providing a base structure having a hydrophilic material presented therewith and one or more electrodes thereon, selectively positioning the device on the vascular structure, wherein the hydrophilic material provides a lubricious surface between the base structure and the vascular structure during positioning, and extending the base structure around at least a portion of the vascular structure and optionally operably coupling the base structure thereto, and selectively re-positioning the device on the vascular structure.

In an embodiment, the method can further include determining an effective position for providing stimulation to the vascular structure before or during selectively positioning or re-positioning the device on the vascular structure, said step of determining an effective position comprising applying an electrical stimulus and observing a response.

In another embodiment, the device can include a belt mechanism having a strap and a buckle, the step of operably coupling the base structure to the vascular structure comprising engaging the strap with the buckle such that the buckle retains at least a portion of the strap and such that the strap can be selectively released to facilitate any re-positioning of the device.

### BRIEF DESCRIPTION OF THE DRAWINGS

FIG. 1 is a schematic view of an upper torso of a human body depicting the major arteries and veins and associated anatomy;

FIG. 2 is a cross-sectional schematic view of a carotid sinus and baroreceptors within a vascular wall of the carotid sinus;

FIG. 3 is a schematic view of the baroreceptors within a vascular wall and a baroreflex system;

FIG. 4 is a schematic view of an upper torso of a human body depicting an intravascular baroreceptor activation system and device disposed near baroreceptors within the vascular wall of the carotid sinus;

FIG. 5 is a close-up view of the carotid sinus of FIG. 4 depicting the intravascular baroreceptor activation device disposed proximate baroreceptors within the vascular wall at the bifurcation of the carotid artery;

FIGS. 6a-6d are cross sectional views of various embodiments of the intravascular baroreceptor activation device of FIG. 5, depicting single or multi-layer coatings presented on an exterior surface thereof;

FIG. 7 is a schematic view of an upper torso of a human body depicting an extravascular baroreceptor activation system and device disposed near baroreceptors within the vascular wall of the carotid sinus;

FIG. 8 is a close-up view of the carotid sinus of FIG. 7 depicting the extravascular baroreceptor activation device disposed proximate baroreceptors within the vascular wall proximate the bifurcation of the carotid artery;

FIGS. 9-12 are schematic views of various embodiments of the extravascular electrode device disposed at the carotid sinus for extravascular electrical activation;

FIGS. 13a and 13b are cross sectional views of wire electrode embodiments of the extravascular baroreceptor activation device of FIGS. 9-12, depicting single or multi-layer coatings presented on an exterior surface thereof;

FIGS. 14a and 14b are cross sectional views of ribbon electrode embodiments of the extravascular baroreceptor activation device of FIGS. 9-12, depicting single or multi-layer coatings presented on an exterior surface thereof;

FIGS. 15a and 15b are cross sectional views of foil electrode embodiments of the extravascular baroreceptor activation device of FIGS. 9-12, depicting single or multi-layer coatings presented on an exterior surface thereof;

FIGS. 16-17 are schematic views of an extravascular electrode device disposed at the carotid sinus for extravascular electrical activation;

FIGS. 18a-18d are cross-sectional views of electrode embodiments of an extravascular baroreceptor activation device, depicting single or multi-layer coatings on the inner and or outer surfaces of the extravascular baroreceptor activation device;

FIG. 19 is a schematic view an extravascular electrical activation device and a tether disposed about the internal carotid artery and common carotid artery, respectively;

FIG. 20 is an elevational view of an extravascular electrode according to a further embodiment;

FIG. 21 is the extravascular electrode of FIG. 20 coupled with the common carotid artery near the carotid bifurcation;

FIG. 22 depicts the electrode of FIG. 20 coupled with the internal carotid artery near the carotid artery bifurcation;

FIG. 23 depicts the electrode of FIG. 22, wherein the carotid artery bifurcation has a different geometry;

FIG. 24 depicts the extravascular electrode of FIG. 20 further including a belt mechanism, wherein the electrode is coupled with the common carotid artery near the carotid artery bifurcation; and

FIG. 25 depicts the extravascular electrode of FIG. 20 further including an aperture and matching protrusion, wherein the electrode is coupled with the common carotid artery near the carotid artery bifurcation.

### DETAILED DESCRIPTION OF THE INVENTION

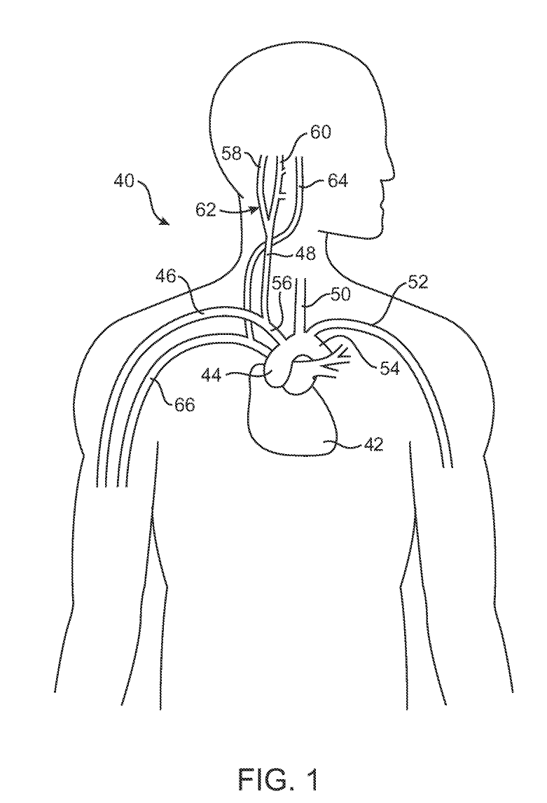

Referring to FIG. 1, an upper torso of a human body 40 is depicted with some of the major arteries and veins of the cardiovascular system. The left ventricle of the heart 42 pumps oxygenated blood up into the aortic arch 44. The right subclavian artery 46, the right common carotid artery 48, the left common carotid artery 50, and the left subclavian artery 52 branch off the aortic arch 44 proximal of the descending thoracic aorta 54. A distinct vascular segment referred to as the brachiocephalic artery 56 connects the right subclavian artery 46 and the right common carotid artery 48 to the aortic arch 44. The right common carotid artery 48 bifurcates into the right external carotid artery 58 and the right internal carotid artery 60 at the right carotid sinus 62. The left carotid artery (not depicted) similarly bifurcates into the left external carotid artery and the left internal carotid artery at the left carotid sinus.

From the aortic arch 44, oxygenated blood flows into the common carotid arteries 48, 50 and the subclavian arteries 46, 52. From the common carotid arteries 48, 50, oxygenated blood circulates through the head and cerebral vasculature and oxygen depleted blood returns to the heart 42 by way of the jugular veins, of which only the right internal jugular vein 64 is depicted. From the subclavian arteries 46, 52, oxygenated blood circulates through the upper peripheral vasculature and oxygen depleted blood returns to the heart 42 by way of the subclavian veins, of which only the right subclavian vein 66 is depicted. The heart 42 pumps the oxygen depleted blood through the pulmonary system where it is re-oxygenated. The re-oxygenated blood returns to the heart 42, which pumps the re-oxygenated blood into the aortic arch 44 as described above. This cycle repeats.

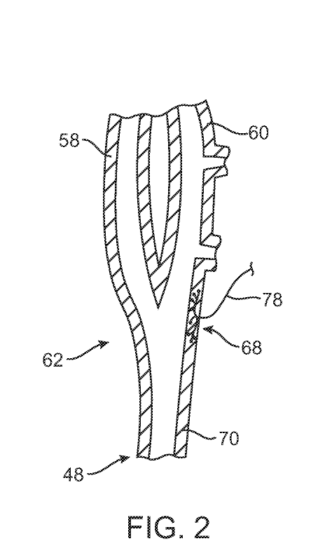

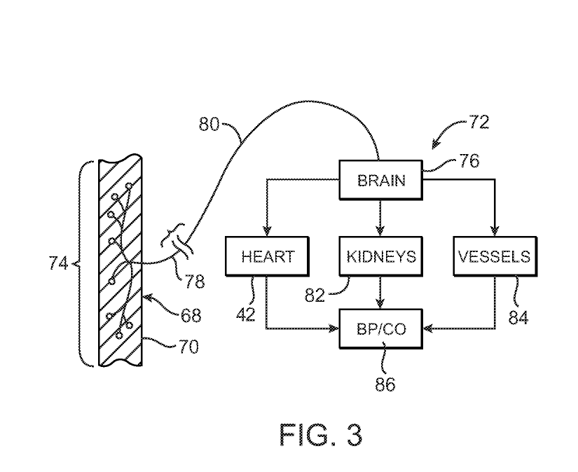

Referring to FIGS. 2 and 3, baroreceptors 68 are located within the arterial walls of the right and left common carotid arteries (near each of the right carotid sinus and left carotid sinus), arterial walls of the aortic arch, subclavian arteries, brachiocephalic artery, and other arteries and veins. The baroreceptors 68 located within the vascular walls of the right common carotid artery 48 near the right carotid sinus 62 will be described herein. Baroreceptors 68 are a type of stretch receptor used by the body to sense blood pressure. An increase in blood pressure causes the arterial wall 70 to stretch and a decrease in blood pressure causes the arterial wall 70 to return to its original size. Such a cycle is repeated with each beat of the heart. Because baroreceptors 68 are located within the arterial wall 70, they are able to sense deformation of the adjacent tissue that is indicative of a change in blood pressure. The baroreceptors 68 located in the right carotid sinus 48, the left carotid sinus, and the aortic arch play a significant role in sensing blood pressure that affects the baroreflex system.

A schematic view of baroreceptors 68 disposed in a generic vascular wall 70 is depicted in FIG. 3 with a schematic flow chart of the baroreflex system 72. Baroreceptors 68 are generally profusely distributed within the vascular walls 70 of the major arteries discussed above to generally form an arbor 74. The baroreceptor arbor 74 comprises a plurality of baroreceptors 68, each of which can transmit baroreceptor signals to the brain 76 via a nerve 78. The baroreceptors 68 are so profusely distributed and arborized within the vascular wall 70 that discrete baroreceptor arbors 74 can be generally indiscernible. Those skilled in the art will recognize that the baroreceptors 68 as depicted in FIGS. 2 and 3 are schematic for illustration and discussion purposes. It will be understood that activation of the baroreflex response for purposes of the present invention can be accomplished by activating a baroreceptor, mechanoreceptors, pressoreceptors, other baroreceptor-like tissue, or nerves emanating therefrom or associated therewith.

Baroreceptor signals are used to activate a number of body systems which collectively can be referred to as the baroreflex system 72. Baroreceptors 68 are connected to the brain 76 via a nerve 78 and the nervous system 80. Thus, the brain 76 is able to detect changes in blood pressure that is indicative of cardiac output 86. If cardiac output 86 is insufficient to meet demand (i.e., the heart is unable to pump sufficient blood), the baroreflex system 72 activates a number of body systems, including the heart 42, kidneys 82, vessels 84, and other organs/tissues. Such activation of the baroreflex system 72 generally corresponds to an increase in neurohormonal activity. Specifically, the baroreflex system 72 initiates a neurohormonal sequence that signals the heart 42 to increase heart rate and increase contraction force in order to increase cardiac output 86, signals the kidneys 82 to increase blood volume by retaining sodium and water, and signals the vessels 84 to constrict to elevate blood pressure. The cardiac, renal and vascular responses increase blood pressure and cardiac output 86, and thus increase the workload of the heart 42. In a patient with heart failure, this further accelerates myocardial damage and exacerbates the heart failure state.

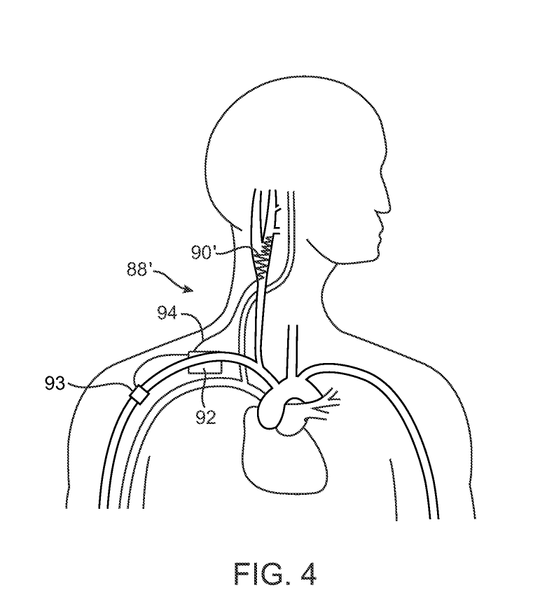

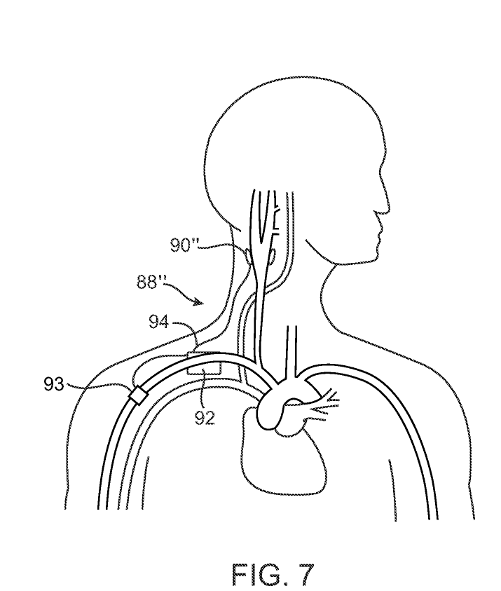

Referring to FIGS. 4 and 7, baroreceptor activation systems 88 (FIG. 4—intravascular 88′ and FIG. 7—extravascular 88″) generally comprises a control system 92, a baroreceptor activation device 90 (intravascular 90′ and/or extravascular 90″), and can comprise one or more sensors 93. The sensors 93 can sense and/or monitor a parameter (e.g., cardiovascular function) indicative of the need to modify the baroreflex system and generates a signal indicative of the parameter. The control system 92 generates a control signal as a function of the received sensor signal. The control signal activates, deactivates or otherwise modulates the baroreceptor activation device 90. Activation of the device 90 can result in activation of the baroreceptors 68 and/or nerves emanating therefrom. Alternatively, deactivation or modulation of the baroreceptor activation device 90 can cause or modify deactivation of the baroreceptors 68 and/or associated nerves.

The baroreceptor activation device 90 can comprise one of a wide variety of devices utilizing mechanical, electrical, thermal, chemical, biological, or other means to activate baroreceptors 68. Thus, when the sensor 93 detects a parameter indicative of the need to modify the baroreflex system 72 activity (e.g., excessive blood pressure), the control system 92 can generate a control signal to modulate the baroreceptor activation device 90 thereby inducing a baroreceptor signal that is perceived by the brain to be apparent excessive blood pressure. When the sensor 93 detects a parameter indicative of normal body function (e.g., normal blood pressure), the control system 92 generates a control signal to modulate (e.g., deactivate) the baroreceptor activation device 90.

The baroreceptor activation device 90 can indirectly activate one or more baroreceptors 68 by stretching or otherwise deforming the vascular wall 70 surrounding the baroreceptors 68. In other embodiments, the baroreceptor activation device 90 can directly activate one or more baroreceptors 68 by changing the electrical, thermal, or chemical environment or potential across the baroreceptors 68. Changing the electrical, thermal, or chemical potential across the tissue surrounding the baroreceptors 68 can also cause the surrounding tissue to stretch or otherwise deform, thus mechanically activating the baroreceptors 68 in addition to electrically, thermally, or chemically activating the baroreceptors 68.

The baroreceptor activation devices 90 described below (intravascular 90′ and extravascular 90″) can generally be suitable for implantation, and are preferably implanted using a minimally invasive percutaneous translumenal approach and/or a minimally invasive surgical approach, depending on whether the device is disposed intravascular, extravascular, or within the vascular wall.

The baroreceptor activation device 90 can be positioned anywhere baroreceptors 68 affecting the baroreflex system 72 are numerous, such as in the heart 42, in the aortic arch 44, in the common carotid arteries 48, 50 near the carotid sinus, in the subclavian arteries 46, 52, or in the brachiocephalic artery 56 or other arteries or veins. The baroreceptor activation device 90 can be implanted such that the device 90 is positioned immediately adjacent the baroreceptors 68. The baroreceptor activation device 90 can be implanted near the right carotid sinus 62 and/or the left carotid sinus and/or the aortic arch 44, where baroreceptors 68 have a significant impact on the baroreflex system 72. For purposes of illustration only, the present embodiments are described with reference to baroreceptor activation device 90 positioned near the carotid sinus 62 in the right carotid artery 48.

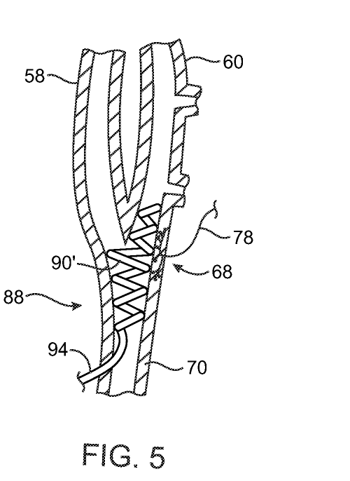

Referring to FIGS. 4 and 5, an intravascular baroreceptor activation system 88′ is depicted. The intravascular baroreceptor activation system 88′ generally comprises an intravascular baroreceptor activation device 90′, a control system 92, a sensor 93, and a lead or line 94 operably coupling the baroreceptor activation device 90′ and control system 92. The intravascular baroreceptor activation device 90′ can comprise, for example, an internal inflatable balloon, an internal deformable coil, or an internal conductive structure (e.g., an electrode). The internal inflatable balloon and internal deformable coil can indirectly activate baroreceptors 68 by stretching or otherwise deforming the vascular wall 70. For example, upon inflation or deflation, the balloon can expand or return to its relaxed geometry such that the baroreceptors 68 and/or the vascular wall 70 are deformed or returned to its nominal state, respectively. With respect to the coil, upon activation or removal of the electrical current, the structure geometry can be changed such that the baroreceptors 68 and/or the vascular wall 70 are removed from or returned to their nominal state. Thus, by selectively changing the balloon or coil structure, the baroreceptors 68 adjacent thereto can be selectively activated or deactivated so that a baroreceptor signal can be induced to effect a change in the baroreflex system of a patient.

The baroreceptor activation device 90′ can also be in the form of an intravascular electrically conductive structure (e.g., electrode). The electrode 90′ can serve the dual purpose of maintaining lumen patency while delivering electrical stimuli. To this end, the electrode 90′ can be implanted utilizing conventional intravascular stent and filter delivery techniques. The electrode 90′ can comprise a geometry enabling blood perfusion therethrough. The electrode 90′ can comprise electrically conductive material, which can be selectively insulated to establish contact with the inside surface of the vascular wall at desired locations, and limit extraneous electrical contact with blood flowing through the vessel and other tissues.

The electrode 90′ can be connected to an electric lead 94, which is operably connected to the control system 92. By selectively activating, deactivating, or otherwise modulating the electrical control signal transmitted to the electrode 90′, electrical energy can be delivered to the vascular wall 70 to activate the baroreceptors 68. As discussed previously, activation of the baroreceptors 68 can occur directly or indirectly. In particular, the electrical signal delivered to the vascular wall 70 by the electrode 90′ can cause the vascular wall 70 to stretch or otherwise deform thereby indirectly activating the baroreceptors 68 disposed therein. Alternatively, the electrical signals delivered to the vascular wall 70 by the electrode 90′ can directly activate the baroreceptors 68 and/or associated nerves. In either case, the electrical signal is delivered to the vascular wall 70 adjacent to the baroreceptors 68. The electrode 90′ can also delivery thermal energy in addition to or in lieu of electrical energy by utilizing a semi-conductive material having a higher resistance such that the electrode 90′ resistively generates heat upon application of electrical energy.

Various further embodiments are contemplated for the electrode 90′, including its design, implanted location, and method of electrical activation. These embodiments are described in detail in U.S. Patent Publication No. 2003/0060857, which is incorporated herein by reference in its entirety.

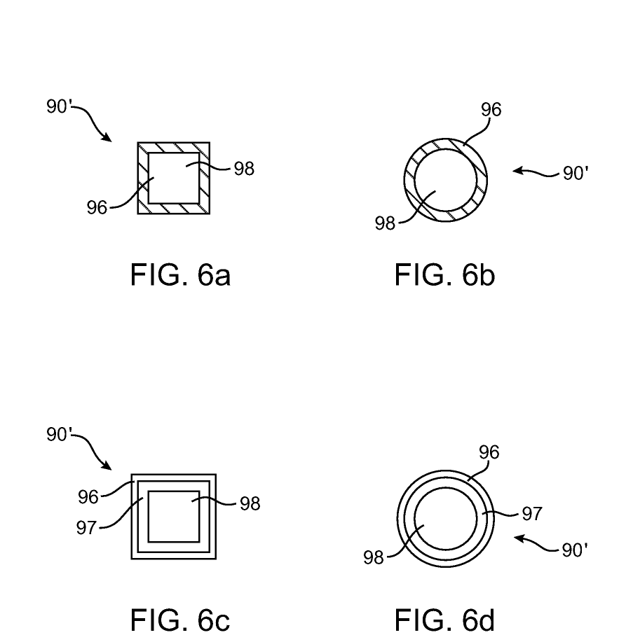

Referring to FIGS. 6a-6d, exemplary cross sections of the intravascular baroreceptor activation device 90′ are depicted. As stated, those skilled in the art will recognize that other cross-sectional electrode 90′ geometries enabling blood perfusion therethrough can be used. For example, the electrodes 90′ can comprise oval wire, rectangular ribbon, or foil formed of an electrically conductive and radiopaque material such as platinum or platinum iridium.

The intravascular baroreceptor activation devices 90′ can comprise a core 98 and one or more single and/or multi-layer coatings disposed thereon. Such coatings can include hydrophilic and hydrophobic coatings 97, 98, respectively. Examples of hydrophilic coatings for use with the intravascular baroreceptor activation device electrode are described in U.S. Pat. Nos. 7,056,533 and 6,706,408 and U.S. Patent Publication No 2003/0215649A1, all of which are incorporated herein by reference in their entirety. Examples of hydrophobic coatings for use with the intravascular baroreceptor activation device electrodes are described in U.S. Pat. No. 7,041,088 and U.S. Patent Publication No. 2006/0105018.

As described above, the intravascular baroreceptor activation device 90′ can be implanted utilizing conventional intravascular stent and filter delivery techniques. Hydrophilic coatings 97 on the exterior surface of the intravascular baroreceptor activation device 90′ can provide a lubricious surface, reducing the amount of friction as the electrode 90′ is inserted into the artery and as the position of the device is adjusted. This can inhibit any damage to the artery and device 90′ during insertion.

The hydrophilic coating 97 can also present a biocompatible and anti-thrombogenic surface inhibiting the formation of scar material and thromboses on or near the surface of the intravascular baroreceptor activation device 90′. By inhibiting such formation, effective blood perfusion through the device 90′ can be maximized and any turbulent blood flow at bifurcation of the carotid artery 62 and through the device 90′ can be reduced.

In addition, inhibition of thromboses or scar material formation on an intravascular baroreceptor activation electrode 90′ can maximize the functionality of the electrode 90′ by not affecting the vessel-electrode interface. As stated, the functionality of the electrode 90′ can depend upon the electrode surface being in good contact with the internal surface of the carotid arteries, such that effective electrical activation of the baroreceptors can occur. By inhibiting or limiting the inflammatory response, and therefore limiting encapsulation of the device 90′ with scarring and/or thromboses, effective vessel-electrode interfacing can be accomplished and/or maintained.

A hydrophobic coating 98 can also be provided on the intravascular baroreceptor activation device between the exterior surface thereof and the hydrophilic coating. The hydrophilic coating adjacent the lumen of the vessel can enable ease of insertion into position within the artery, while the hydrophobic layer can enable the promotion of long term adhesion once the intravascular baroreceptor activation device is properly positioned.

Anti-inflammatory agents can also be included in one or more of the hydrophilic and/or hydrophobic coatings (e.g., steroid eluting electrodes), such as those described in U.S. Pat. No. 4,711,251, U.S. Pat. No. 5,522,874, and U.S. Pat. No. 4,972,848, all of which are incorporated herein by reference in their entirety. Such agents can reduce tissue inflammation at the chronic interface between the device (e.g., electrodes) 90′ and the vascular wall tissue, thereby increasing the efficiency of stimulus transfer, reducing power consumption, and maintaining activation efficiency.

Referring to FIGS. 6a and 6b, a hydrophilic coating 96 is disposed on the core 98 of the device 90′. In this embodiment, the hydrophilic coating 96 can provide a lubricious surface, reducing the amount of friction as the device 90′ is positioned in the artery. This can inhibit any damage to the artery and device 90′ during placement. The hydrophilic coating 96 can also inhibit the formation of thromboses or scar material on the inner lumen of the artery or outer surface of the device.

Referring to FIGS. 6c and 6d, a hydrophobic coating 97 can be disposed intermediate the hydrophilic coating 96 and the core 98. In this embodiment, the hydrophilic coating 96 can provide a lubricious surface, reducing the amount of friction as the device 90′ is positioned in the artery. This can inhibit any damage to the artery and device 90′ during placement. The hydrophobic layer 97 can enable the promotion of long term adhesion once the intravascular baroreceptor activation device 90′ is properly positioned.

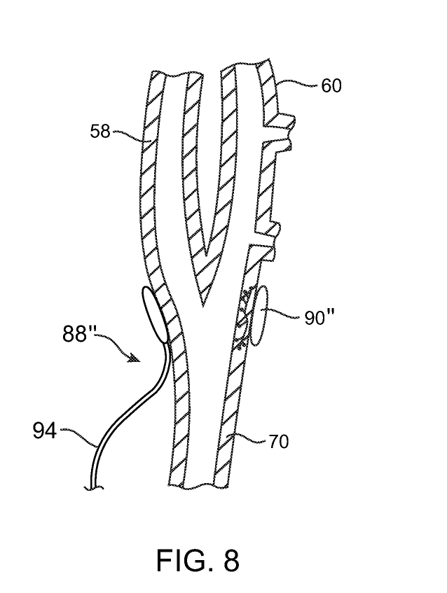

Referring to FIGS. 7 and 8, an extravascular baroreceptor activation system 88″ generally comprises an extravascular baroreceptor activation device 90″, a control system 92, a sensor 93, and a lead or line 94 operably coupling the baroreceptor activation device 90″ and control system 92. An extravascular baroreceptor activation device 90″ can comprise, for example, an external pressure cuff, an external deformable coil, an external flow regulator, a transducer, a fluid delivery device, a magnetic device, an external conductive structure (e.g., an electrode), or an external Peltier device. As with the intravascular baroreceptor activation device 90″, by selectively activating, deactivating, or otherwise modulating the extravascular baroreceptor activation device 90″, a baroreceptor signal can be induced to effect a change in the baroreflex system of a patient.

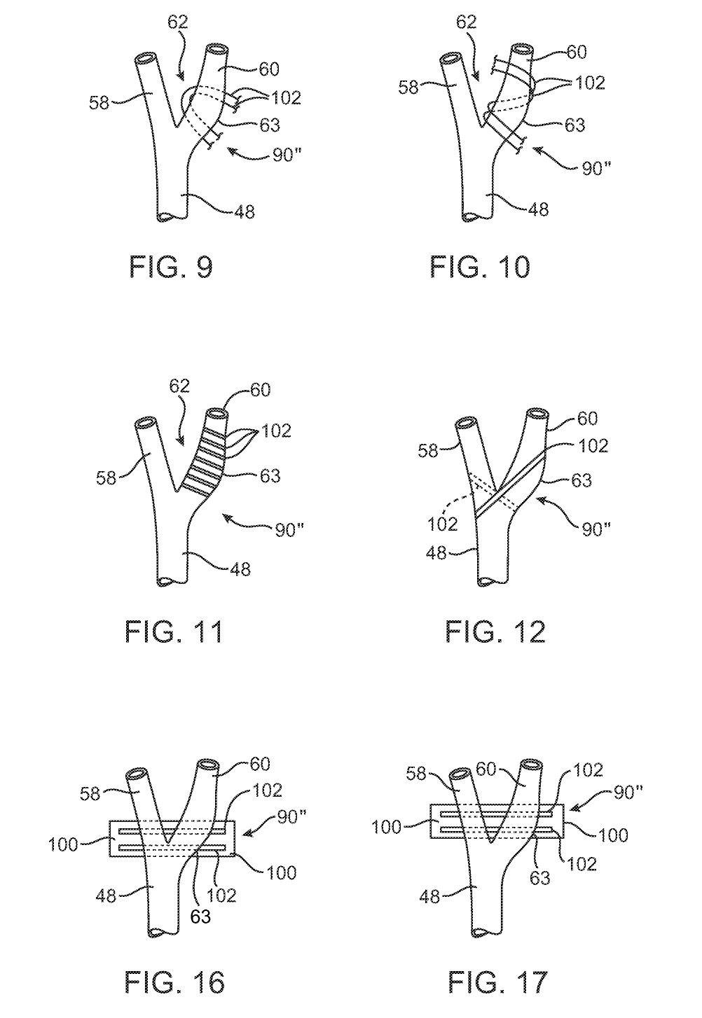

Referring to FIGS. 9-12, 16, and 17, various embodiments of the extravascular baroreceptor activation device 90″ in the form of electrodes disposed at the carotid artery 48 are depicted. The location of the carotid sinus 62 can be identified by a landmark sinus bulge 63, which is typically located on the internal carotid artery 60 just distal of the bifurcation of the common carotid artery 48 into the external carotid artery 58 and the internal carotid artery 60. The carotid sinus 62, and in particular the sinus bulge 63 of the carotid sinus 62, can contain a relatively high density of baroreceptors 68 in the vascular wall 70. For this reason, it can be desirable to position the extravascular electrode activation device 90″ on and/or around the sinus bulge 63 to maximize baroreceptor 68 responsiveness and to minimize extraneous tissue stimulation.

The electrodes 90″ are depicted schematically for purposes of illustrating various positions of the electrodes 90″ on and/or around the carotid sinus 62 and the sinus bulge 63. In each of the embodiments, the electrodes 90″ can be monopolar, bipolar, or tripolar (anode-cathode-anode or cathode-anode-cathode sets). In addition to the embodiments depicted and described herein, further extravascular electrode 90″ designs are described in U.S. Patent Publication No. 2003/0060857, U.S. Patent Publication No. 2004/0010303, U.S. Patent Publication No. 2006/0004430, U.S. Patent Publication No. 2006/0111626, and co-pending U.S. Patent Application No. 60/805,707, entitled “IMPLANTABLE ELECTRODE ASSEMBLY UTILIZING A BELT MECHANISM FOR SUTURELESS ATTACHMENT,” all of which are incorporated by reference in their entirety.

Referring to FIG. 9, specifically, the extravascular electrical activation devices 90″ can extend around a portion or the entire circumference of the carotid sinus 62 in a circular fashion. In FIG. 10, the electrodes 102 of the extravascular electrical activation device 90″ extend around a portion or the entire circumference of the carotid sinus 62 in a generally helical fashion. In the helical arrangement, the electrodes 102 can wrap around the sinus 62 any number of times to establish the desired contact and coverage. In the arrangement as depicted in FIG. 11, a single pair of electrodes 102 or multiple pairs of electrodes 102 can wrap around the sinus 62 multiple times to establish further contact and coverage.

The electrode pairs 102 can extend from a point proximal of the sinus 62 or bulge 63 to a point distal of the sinus 62 or bulge 63 to ensure activation of baroreceptors 68 throughout the sinus 62 region. The electrodes 102 can be connected to a single channel or multiple channels. The plurality of electrode pairs 102 can be selectively activated for purposes of targeting a specific area of the sinus 62 to increase baroreceptor 68 responsiveness, or for purposes of reducing the exposure of tissue areas to activation to maintain baroreceptor 68 responsiveness during a long term.

In FIG. 12, the electrodes 102 extend around the entire circumference of the sinus 62 in a criss-cross fashion. The criss-cross arrangement of the electrodes 102 enables contact with both the internal and external carotid arteries 58, 60 around the carotid sinus 62.

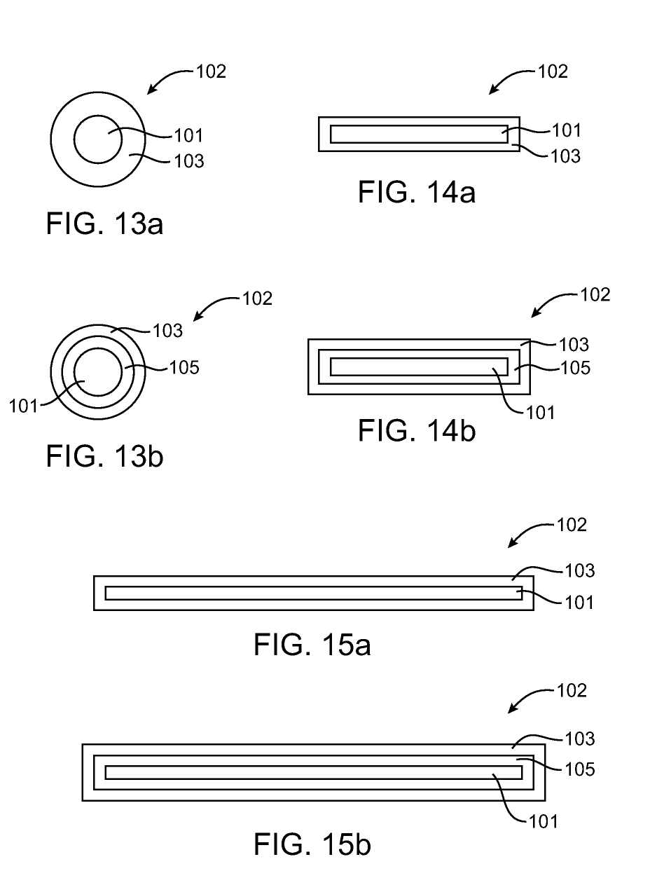

Referring to FIGS. 13a, 13b, 14a, 14b, 15a, and 15b, various exemplary cross sections of the extravascular baroreceptor activation devices 90″ of FIGS. 9-12 are depicted. The electrodes 102 can comprise round wire (FIGS. 13a and 13b), rectangular ribbon (FIGS. 14a and 14b), or foil (FIGS. 15a and 15b), formed of an electrically conductive material, that can also be radiopaque, such as platinum or platinum iridium. Those skilled in the art will recognize that other cross-sectional electrode 90″ geometries can be used.

The extravascular baroreceptor activation electrodes 102 can comprise a core 102 and one or more single and/or multi-layer coatings disposed thereon. Such coatings can include hydrophilic coatings 103 and hydrophobic coatings 105. Hydrophilic coatings 103 on the surface of the electrodes 102 can provide a lubricious surface, reducing the amount of friction as the electrode 90″ is positioned onto the artery proximate the baroreceptors. This can inhibit any damage to the artery and device 90″ during implantation.

The hydrophilic coating 103 can also present a biocompatible surface that facilitates the inhibition of the formation of scar material on or near the surface of the extravascular baroreceptor activation device 90″. Reducing or inhibiting such formation on an extravascular baroreceptor activation electrode 90″ can maximize the functionality of the baroreceptor activation device 90″ by not affecting the vessel-electrode interface. As discussed above, the functionality of the electrode 90″ can depend upon the electrode 90″ being in good contact with the internal surface of the vessel, such as the carotid arteries, in order that effective electrical activation of the baroreceptors can occur so that a baroreceptor signal can be induced to effect a change in the baroreflex system of a patient. By reducing or inhibiting the inflammatory response, and therefore encapsulation of the device 90″ with scarring and/or thromboses, a more effective vessel-electrode interface can be accomplished and/or maintained.

A hydrophobic coating 105 can also be provided on the electrode 102 of the extravascular baroreceptor activation device 90″ between the exterior surface of the core 101 and the hydrophilic coating 103. In this embodiment, the hydrophilic coating 103 adjacent the interior surface of the vessel can enable ease of positioning onto position in the artery. The hydrophobic layer 105 intermediate the core 101 and the hydrophilic coating 103 can enable the promotion of long term adhesion once the intravascular baroreceptor activation device 90″ is properly positioned.

Anti-inflammatory agents can be included in one or more of the hydrophilic and/or hydrophobic coatings (e.g., steroid eluting electrodes), such as those described in U.S. Pat. No. 4,711,251, U.S. Pat. No. 5,522,874, and U.S. Pat. No. 4,972,848, all of which are incorporated herein by reference in their entirety. Such agents can reduce tissue inflammation at the chronic interface between the device 90″ (e.g., electrodes) and the vascular wall tissue, thereby increasing the efficiency of stimulus transfer, reducing power consumption, and maintaining activation efficiency.

Referring to FIGS. 16-17, the extravascular electrical activation devices 90″ are depicted to include a substrate or base structure 100, which can encapsulate or support the electrode pairs 102 and can provide a means for attachment to the sinus as described in more detail hereinafter.

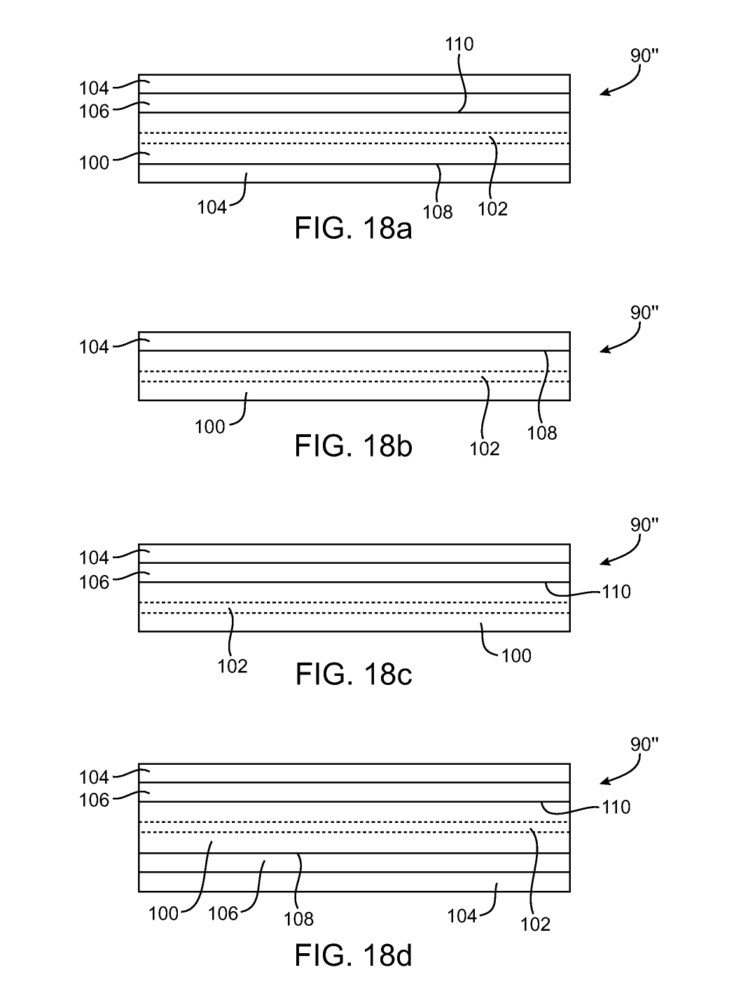

Referring to FIGS. 18a-18d, various exemplary cross sections of the extravascular baroreceptor activation device 90″ of FIGS. 16-17 are depicted. Each embodiment of the extravascular baroreceptor activation device 90″ can comprise a base 100 and at least one of a hydrophilic coating 104 and/or a hydrophobic coating 106 on an interior surface 110 and/or exterior surface 108 thereof. Examples of hydrophilic coatings 104 for use with the extravascular electrode activation device 90″ are described in U.S. Pat. Nos. 7,056,533 and 6,706,408 and U.S. Patent Publication No 2003/0215649A1, all of which are incorporated herein by reference in their entirety. Examples of hydrophobic coatings for use with the extravascular electrode activation device 90″ are described in U.S. Pat. No. 7,041,088 and U.S. Patent Publication No. 2006/0105018.

Referring to FIG. 18a, a hydrophilic coating 104 is disposed on the exterior surface 108 of the base 100. A hydrophobic coating 106 is disposed on the interior surface 110 of the base. A second hydrophilic coating 104 is then disposed on the hydrophobic coating 106. In this embodiment, the hydrophilic coating 104 on the hydrophobic coating 106 can provide a lubricious surface, reducing the amount of friction as the device 90″ is positioned on the artery. This can inhibit any damage to the artery and device 90″ during placement. The hydrophobic layer 106 can enable the promotion of long term adhesion once the extravascular baroreceptor activation device 90″ is properly positioned. The hydrophilic coating 104 on the exterior surface 108 of the electrode 102 can reduce or inhibit the formation of scar material on the exterior of the artery or interior surface of the electrode that otherwise could contract the electrode on the artery causing a false parameter indicative of the need to modify the baroreflex system activity causing the control system to generate a control signal activating the baroreceptor activation device to induce a baroreceptor signal that is perceived by the brain to be apparent excessive blood pressure.

Referring to FIG. 18b, a hydrophilic coating 104 is disposed on the exterior surface 108. In this embodiment, the hydrophilic coating 104 adjacent the exterior surface of the vessel can provide a lubricious surface, reducing the amount of friction as the device 90″ is positioned on the artery. This can inhibit any damage to the artery and device 90″ during placement.

Referring to FIG. 18c, a hydrophobic coating 106 is disposed on the interior surface 110. A second hydrophilic coating 104 is then disposed on the hydrophobic coating 106. In this embodiment, the hydrophilic coating 104 on the hydrophobic coating 106 adjacent the exterior surface of the vessel can provide a lubricious surface, reducing the amount of friction as the device 90″ is positioned on the artery. This can inhibit any damage to the artery and device 90″ during placement. The hydrophobic layer 106 can enable the promotion of long term adhesion once the extravascular baroreceptor activation device 90″ is properly positioned.

Referring to FIG. 18d, hydrophobic coatings 106 are disposed on the interior and exterior surfaces 110, 108. Hydrophilic coatings 104 are then disposed on the hydrophobic coating 106.

The base 100 in the various embodiments can comprise insulative properties, or insulation, such that the stimulation is directionally provided toward the vascular structure.

From the foregoing discussion with reference to FIGS. 9-12, 16, and 17, one skilled in the art will recognize that numerous arrangements can be used for the electrodes of the extravascular activation device. In each of the examples above, the electrodes are generally wrapped around a portion of the carotid structure, requiring deformation of the electrodes from their relaxed geometry (e.g., straight). To reduce such deformation, the electrodes and/or the base can comprise a relaxed geometry substantially conformable to the shape of the carotid anatomy at the point of attachment. In other words, the electrodes and the base structure or backing can be pre-shaped to generally conform to the vessel anatomy in a substantially relaxed state. Alternatively, the electrodes can have a geometry and/or orientation that reduce the amount of electrode strain. Optionally, the base can comprise elasticity or otherwise be stretchable to facilitate wrapping of and conforming to the carotid sinus or other vascular structure.

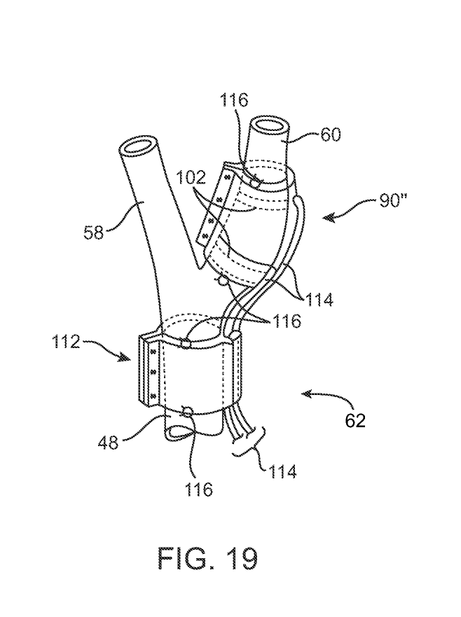

FIG. 19 schematically depicts an extravascular electrical activation device 90″ including a support collar or anchor 112. In this embodiment, the activation device 90″ is wrapped around or otherwise coupled to the internal carotid artery 60 at the carotid sinus 62, and the support collar 112 is wrapped around or otherwise coupled to the common carotid artery 48. The activation device 90″ is connected to the support collar 112 by cables 114, which act as a loose tether. With this arrangement, the collar 112 isolates the activation device 90″ from movements and forces transmitted by the cables 114 proximal of the support collar 112, such as can be encountered by movement of the control system 92. As an alternative to support collar 112, a strain relief (not depicted) can be connected to the base of the activation device 90″ at the juncture between the cables 112 and the base 100. With either approach, the position of the device 90″ relative to the carotid anatomy can be better maintained despite movements of other parts of the system.

In this embodiment, the base 100 of the activation device can comprise molded tube, a tubular extrusion, or a sheet of material wrapped into a tube shape utilizing a suture flap with sutures 116 as depicted. The base 100 can be formed of a flexible and biocompatible material such as silicone, which can be reinforced with a flexible material such as polyester fabric available under the trade name Dacron® to form a composite structure. The inside diameter of the base 100 can correspond to the outside diameter of the carotid artery at the location of implantation, for example 6 to 8 mm. The wall thickness of the base 100 can be very thin to maintain flexibility and a low profile, for example less than 1 mm. If the device 90″ is to be disposed about a sinus bulge 62, a correspondingly shaped bulge can be formed into the base 100 for added support and assistance in positioning.

The electrodes 102 (depicted in phantom lines) can comprise round wire, rectangular ribbon, or foil, formed of an electrically conductive and radiopaque material such as platinum or platinum iridium. The electrodes 102 can be molded into the base 100 or adhesively connected to the inside diameter thereof, leaving a portion of the electrode 102 exposed for electrical connection to carotid tissues. The electrodes 102 can encompass less than the entire inside circumference (e.g., 300°) of the base 100 to avoid shorting. The electrodes 102 can have any of the shapes and arrangements described previously.

The support collar 112 can be formed similarly to base 100. For example, the support collar 112 can comprise molded tube, a tubular extrusion, or a sheet of material wrapped into a tube shape utilizing a suture flap with sutures 116 as depicted. The support collar 112 can be formed of a flexible and biocompatible material such as silicone, which can be reinforced to form a composite structure. The cables 114 are secured to the support collar 112, leaving slack in the cables 114 between the support collar 112 and the activation device

Those skilled in the art will recognize that it can be desirable to secure the activation device 90″ to the vascular wall 70 using sutures or other fixation means. For example, sutures can be used to maintain the position of the electrical activation device 90″ relative to the carotid anatomy (or other vascular site containing sites to be activated). Such sutures can be connected to base 100, and pass through all or a portion of the vascular wall 70. For example, the sutures can be threaded through the base structure, through the adventitia of the vascular wall, and tied. If the base 100 comprises a patch or otherwise partially surrounds the carotid anatomy, the corners and/or ends of the base 100 can be sutured, with additional sutures evenly distributed therebetween. In order to minimize the propagation of a hole or a tear through the base structure, a reinforcement material such as polyester fabric can be embedded in the silicone material. In addition to sutures, other fixation means can be employed such as, for example, staples or a biocompatible adhesive.

The inner surfaces of the extravascular baroreceptor activation device 90″ and/or the collar can comprise a hydrophilic coating and/or hydrophobic coating thereon. The hydrophilic coatings on the interior surface of the extravascular baroreceptor activation device 90″ and/or the collar can provide a lubricious surface, reducing the amount of friction as the device 90″ and/or the collar are positioned on the artery. This can inhibit any damage to the artery, device 90″, and collar during placement. The coating can also provide a biocompatible surface inhibiting the formation of scar material on the exterior of the artery or interior surface of the electrode 90″ and collar that otherwise could contract the electrode and the collar on the artery causing a false parameter indicative of the need to modify the baroreflex system activity causing the control system to generate a control signal activating the baroreceptor activation device to induce a baroreceptor signal that is perceived by the brain to be apparent excessive blood pressure.

In addition, by inhibiting the formation of scar material on the electrode, the functionality of the activation device 90″ can be maximized by optimizing the electrical characteristics of the vessel-electrode interface. As has been described, when using externally positioned electrodes, the functionality of the device can depend upon the inner surface of the device being in good contact with the exterior surface of the carotid arteries, such that effective activation of the baroreceptors can occur.

The hydrophobic coating can also be provided to enable the promotion of long term adhesion of the extravascular activation device 90″ and/or the collar 112 once they are properly positioned.

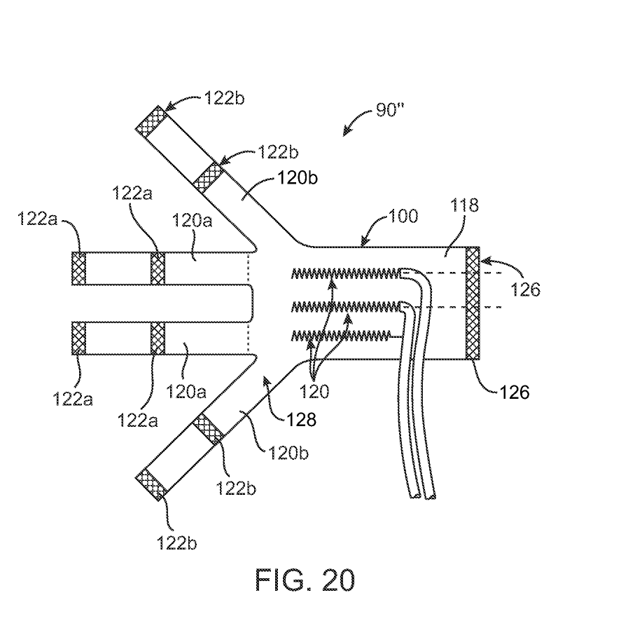

Referring now to FIGS. 20-25, a further electrode embodiment is depicted. Electrode 90″ comprises a base 100, which can be elastic and formed silicone or other elastomeric material, having an electrode-carrying surface 118 and a plurality of attachment tabs 120 extending from the electrode-carrying surface 118. The attachment tabs 120 can be formed from the same material as the electrode-carrying surface 118 or formed from other elastomeric materials. In the latter case, the base 100 will be molded, stretched, or otherwise assembled from the various pieces. In the illustrated embodiment, the attachment tabs 120 are formed integrally with the remainder of the base 100, i.e., being cut from a single sheet of the elastomeric material.

The geometry of the electrode 90″, and in particular the geometry of the base 100, is selected to enable a number of different attachment modes to the blood vessel. In particular, the geometry of the device of FIG. 20 is intended to enable attachment to various locations on the carotid arteries at or near the carotid sinus 62 and the bifurcation of the common carotid 48 into internal and external carotid arteries 58, 60.

A number of reinforcement regions 122 are attached to different locations on the base 100 to enable suturing, clipping, stapling, or other fastening of the attachment tabs 120 to each other and/or the electrode-carrying surface 118 of the base 100. In an embodiment intended for attachment at or around the carotid sinus 62, a first reinforcement strip 124 is provided over a first end 126 of the base 100 opposite to a second end 128 which carries the attachment tabs 120. Pairs of reinforcement strips 130 and are provided on each of the axially aligned attachment tabs 120a, while similar pairs of reinforcement strips 130 are provided on each of the transversely angled attachment tabs 120b. In the illustrated embodiment, all attachment tabs 120 will be provided on one side of the base 100, preferably emanating from adjacent corners of the rectangular electrode-carrying surface 118.

The structure of electrode 90″ enables the surgeon to implant the electrode 90″ so that the electrodes 102 (which can be stretchable, flat-coil electrodes) are located at a location relative to the target baroreceptors. The preferred location can be determined, for example, as described in U.S. Pat. No. 6,850,801, which is incorporated herein by reference in its entirety.

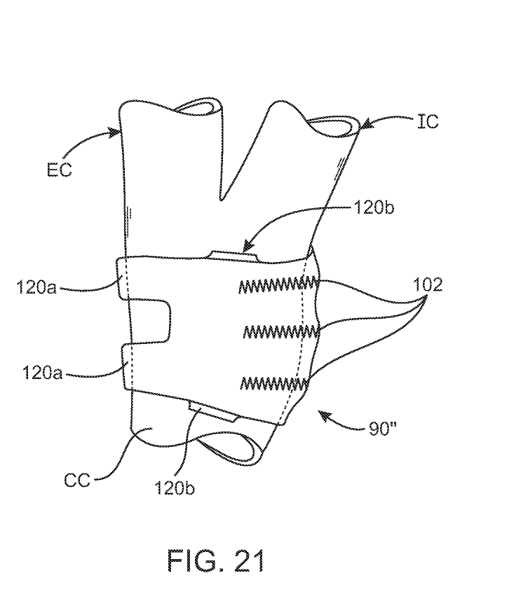

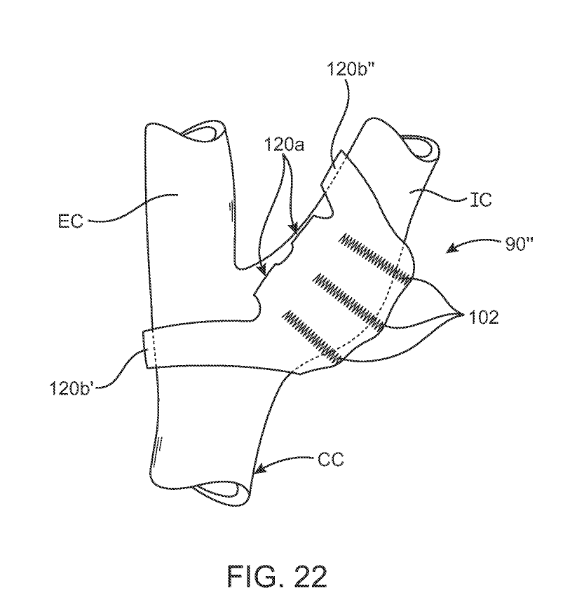

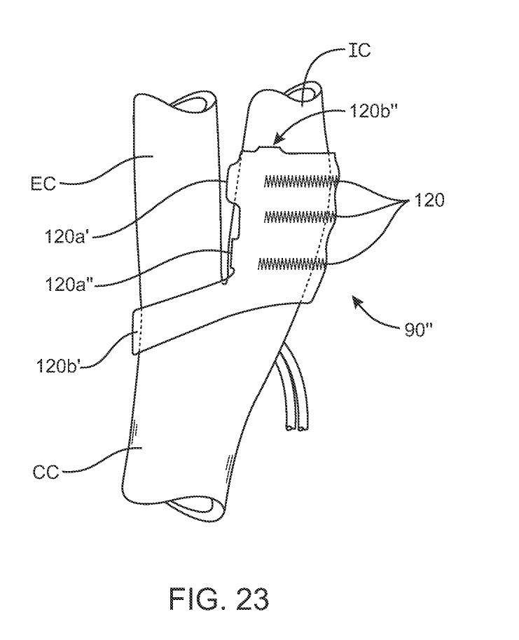

Once the selected location for the electrodes 102 of the electrode assembly 90″ is determined, the surgeon can position the base 100 so that the electrodes 102 are located appropriately relative to the underlying tissue. Thus, the electrodes 102 can be positioned over the common carotid artery CC as depicted in FIGS. 21, 24, and 25, or over the internal carotid artery IC, as depicted in FIGS. 22-23. In FIG. 21, the assembly can be attached by stretching the base 100 and axially aligned attachment tabs 120a over the exterior of the common carotid artery CC. The reinforcement tabs 122a can then be secured to the reinforcement strip 126, either by suturing, stapling, fastening, gluing, welding, or other known mechanisms. Attachment tabs 120b can be cut off at their bases.

In other cases, the bulge of the carotid sinus 62 and the baroreceptors can be located differently with respect to the carotid bifurcation. For example, as depicted in FIGS. 22-23, the receptors can be located further up the internal carotid artery IC so that the placement of electrode 90″ over the exterior of the common carotid artery CC as depicted in FIG. 21 will generally not be as effective. The electrode 90″, however, can still be successfully attached by utilizing the transversely angled attachment tabs 120b rather than the central or axial tabs 120a. As depicted in FIG. 22, the lower tab 120b′ is wrapped around the common carotid artery CC, while the upper attachment tab 120b″ is wrapped around the internal carotid artery IC. The axial attachment tabs 120a will usually be cut off, although either of them could in some instances also be wrapped around the internal carotid artery IC. Again, the tabs 120b used can be stretched and attached to reinforcement strip 126, as generally described above.

Referring to FIG. 23, in instances where the carotid bifurcation has less of an angle, the assembly can be attached using the upper axial attachment tab 120a′ and be lower transversely angled attachment tab 120b′. Attachment tabs 120a″, 120b″ can be cut off. The elastic nature of the base 100 and the stretchable nature of the electrodes 102 enable the desired conformance and secure mounting of the electrode 90″ over the carotid sinus 62. Those skilled in the art will recognize that these and/or similar structures can also be useful for mounting electrodes at other locations in the vascular system.

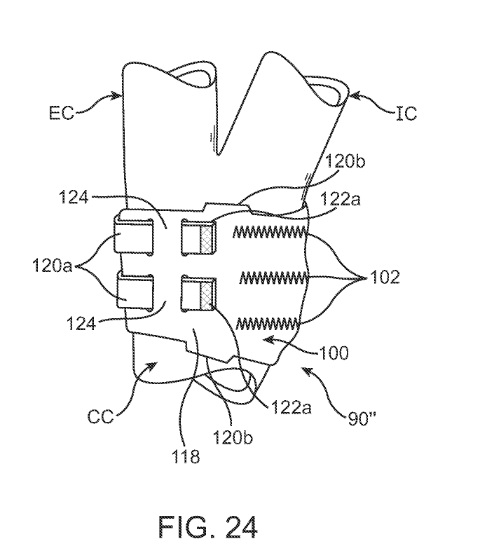

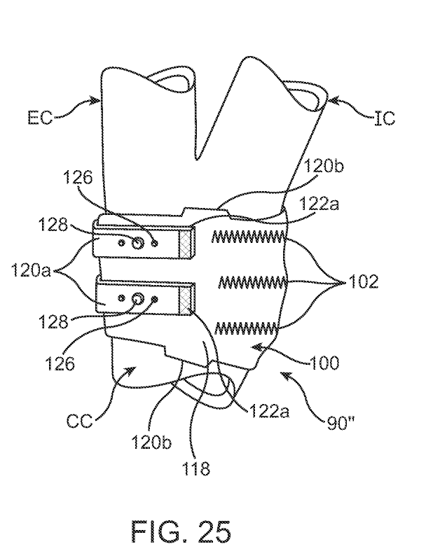

Referring to FIGS. 24 and 25, mechanisms can be included on the device 90″ to facilitate the electrode attachment to the carotid artery. For example, in FIG. 24, a loop or slot 124 can be included on the rectangular electrode-carrying surface 118. Once the preferred location for the electrodes 102 of the electrode assembly 90″ is determined, the surgeon can position the base 100 so that the electrodes 102 are located appropriately relative to the underlying baroreceptors. Thus, the electrodes 102 can be positioned over the common carotid artery CC, the assembly can be attached by stretching the base 100 and axially aligned attachment tabs 120a over the exterior of the common carotid artery CC. The axially aligned attachment tabs 120a can then be looped through the loop or slot 124. Attachment tabs 120b can be cut off at their bases.

Referring to FIG. 25, an aperture 126 and projection 128 can be included on the rectangular electrode-carrying surface 118. Once the preferred location for the electrodes 102 of the electrode assembly 90″ is determined, the surgeon can position the base 100 so that the electrodes 102 are located appropriately relative to the underlying baroreceptors. Thus, the electrodes 102 can be positioned over the common carotid artery CC, the assembly can be attached by stretching the base 100 and axially aligned attachment tabs 120a over the exterior of the common carotid artery CC. The aperture 126 and projection 128 on the axially aligned attachment tabs 120a can then be coupled. Attachment tabs 120b can be cut off at their bases.

As discussed above, the inner surfaces of the extravascular baroreceptor activation device 90″ can comprise a hydrophilic coating and/or hydrophobic coating thereon. The hydrophilic coatings on the interior surface of the extravascular baroreceptor activation device 90″ and/or the collar can provide a lubricious surface, reducing the amount of friction as the device 90″ and/or the collar are positioned on the artery. This can inhibit any damage to the artery, device 90′, and collar during placement. The coating can also provide a biocompatible surface inhibiting the formation of scar material on the exterior of the artery or interior surface of the electrode 90″ and collar, which otherwise could contract the electrode and the collar on the artery causing a false parameter indicative of the need to modify the baroreflex system activity causing the control system to generate a control signal activating the baroreceptor activation device to induce a baroreceptor signal that is perceived by the brain to be apparent excessive blood pressure.

The hydrophobic coating can also be provided to enable the promotion of long term adhesion of the extravascular baroreceptor activation device 90″ once it is properly positioned.

In addition, by inhibiting the formation of scar material on the electrode, the functionality of the baroreceptor activation device 90″ can be maximized by optimizing the electrical characteristics of the vessel-electrode interface. As has been described, when using externally positioned electrodes, the functionality of the device can depend upon the inner surface of the device being in good contact with the exterior surface of the carotid arteries, such that effective activation of the baroreceptors can occur.

Although the devices herein have been described with reference to particular embodiments, one skilled in the art will recognize that changes can be made in form and detail. Specifically, while the devices and methods herein have been depicted and described with reference to activating, deactivating, or otherwise modulating a device to provide stimulation to a vascular structure for purposes of eliciting a baroreflex response, those skilled in the art will recognize that the methods and devices herein can be used for other types of stimulation directed to a vessel wall for the purposes of eliciting a physiologic response. Therefore, the illustrated embodiments should be considered in all respects as illustrative and not restrictive. Any incorporation by reference of documents above is limited such that no subject matter is incorporated that is contrary to the explicit disclosure herein.

What is claimed is:

1. A device selectively positionable on a vascular structure for providing stimulation to the vascular structure for purposes of eliciting a physiologic response, the device comprising:
a base structure comprising inner and outer surfaces and a hydrophilic material presented on at least a portion of the inner surface presenting a lubricious surface for selectively positioning the device on a vascular structure; and
an electrode structure presented with the base structure operable to provide stimulation to the vascular structure, wherein the base structure and electrodes are configured to conform to at least a portion of the vascular structure to maintain an intimate vascular structure-electrode interface.

2. The device of claim 1, wherein the vascular structure comprises a carotid artery and the base structure comprises a length enabling the base structure to extend substantially around the carotid artery for conforming thereto.

3. The device of claim 1, wherein the base structure comprises insulation, such that the stimulation is directionally provided toward the vascular structure.

4. The device of claim 1, further comprising an anti-inflammatory agent presented with the hydrophilic material.

5. The device of claim 1, wherein hydrophilic material is further presented on the outer surface of the base structure.

6. The device of claim 5, further comprising an anti-inflammatory agent presented with the hydrophilic material.

7. The device of claim 1, further comprising a hydrophobic material presented intermediate the inner surface and the hydrophilic material presented on the inner surface.

8. The device of claim 1, further comprising:
a hydrophobic material presented intermediate the inner surface and the hydrophilic material presented on at least a portion of the inner surface and wherein the hydrophilic material is further presented on the outer surface of the base structure; and
an anti-inflammatory agent presented with the hydrophilic material wherein the base structure comprises insulation, such that the stimulation is directionally provided toward the vascular structure.

9. The method of claim 8, wherein the vascular structure comprises a carotid artery and the base structure comprises a length enabling the base structure to extend substantially around the carotid artery conforming thereto.

10. A method of providing stimulation to a vascular structure for purposes of eliciting a physiologic response, the method comprising:
providing a device comprising a base structure having a hydrophilic material presented therewith and an electrode structure thereon;
selectively positioning the device on the vascular structure, wherein the hydrophilic material provides a lubricious surface for effecting movement of the device with respect to the vascular structure during positioning;
extending the base structure around at least a portion of the vascular structure and selectively re-positioning the device on the vascular structure; and
activating, deactivating, or otherwise modulating the device to provide stimulation to the vascular structure for purposes of eliciting the physiologic response.

11. The method of claim 10, further comprising determining an effective position for providing stimulation to the vascular structure before or during the step of selectively positioning the device on the vascular structure.

12. The method of claim 11, wherein determining the effective position comprises applying a stimulus and observing a response.

13. The method of claim 10, wherein the device comprises a belt mechanism comprising a strap and a buckle, the step of operably coupling the base structure to the vascular structure comprising engaging the strap with the buckle, such that the buckle retains at least a portion of the strap.

14. The method of claim 10, wherein the step of operably coupling the base structure to the vascular structure comprises suturing the base structure to at least one of the vascular structure and the nerves associated with the vascular structure.

15. The method of claim 10, wherein the vascular structure comprises a carotid artery having one or more baroreceptors therein, the step of selectively positioning the device on the vascular structure comprising determining the location of the one or more baroreceptors and effecting movement of the device such that the device is proximate the one or more baroreceptors.

16. The method of claim 10, wherein the base structure further comprises a hydrophobic material presented therewith, the method further comprising enabling adhesion between the vascular structure and the hydrophobic material.

17. The method of claim 10, wherein the step of extending the base structure around at least a portion of the vascular structure further comprises operably coupling the base structure to at least a portion of the vascular structure.

18. The method of claim 10, further comprising determining an effective position for providing stimulation to the vascular structure before or during the step of selectively positioning the device on the vascular structure, wherein the step of determining an effective position comprises applying a stimulus and observing a response,
wherein the base structure further comprises a hydrophobic material presented therewith, the method further comprising enabling adhesion between the vascular structure and hydrophobic material, and
wherein the device comprises a belt mechanism comprising a strap and a buckle, the step of operably coupling the base structure to the vascular structure comprising engaging the strap with the buckle such that the buckle retains at least a portion of the strap.

19. The method of claim 18, wherein the vascular structure comprises a carotid artery having one or more baroreceptors therein, the step of selectively positioning the device on the vascular structure comprising determining the location of the one or more baroreceptors and effecting movement of the device such that the device is proximate the one or more baroreceptors.

20. A method of selectively positioning a device on a vascular structure for providing stimulation to the vascular structure for purposes of eliciting a physiologic response, the method comprising:
providing a base structure having a hydrophilic material presented therewith and one or more electrodes thereon;
selectively positioning the device on the vascular structure, wherein the hydrophilic material provides a lubricious surface between the base structure and the vascular structure during positioning; and
extending the base structure around at least a portion of the vascular structure and selectively re-positioning the device on the vascular structure and operably coupling the base structure thereto.

21. The method of claim 20, further comprising determining an effective position for providing stimulation to the vascular structure before or during selectively positioning the device on the vascular structure.

22. The method of claim 21, wherein said step of determining an effective position comprises applying a stimulus and observing a response.

23. The method of claim 20, wherein the device comprises a belt mechanism comprising a strap and a buckle, the step of operably coupling the base structure to the vascular structure comprising operably engaging the strap with the buckle such that the buckle retains at least a portion of the strap.

24. The method of claim 20, wherein the vascular structure comprises a carotid artery having one or more baroreceptors therein, the step of selectively positioning the device on the vascular structure comprising determining the location of the one or more baroreceptors and effecting movement of the device such that the device is proximate one or more baroreceptors.

25. The method of claim 24, wherein the step of determining the location of the one or more baroreceptors comprises measuring the efficacy of a test stimulation.

26. The method of claim 20, further comprising determining an effective position for providing the stimulation to the vascular structure before or during the step of selectively positioning the device on the vascular structure, wherein the step of determining the effective position comprises applying a stimulus and observing a response,
wherein the vascular structure comprises a carotid artery having baroreceptors therein, the step of selectively positioning the device on the vascular structure comprising determining the location of one or more baroreceptors and effecting movement of the device such that the device is proximate one or more baroreceptors,
wherein the base structure further comprises a hydrophobic material presented therewith, the method further comprising enabling adhesion between the vascular structure and hydrophobic material, and
wherein the device comprises a belt mechanism comprising a strap and a buckle, the step of operably coupling the base structure to the vascular structure comprising engaging the strap with the buckle such that the buckle retains at least a portion of the strap.

27. A method of directing stimulation to a vessel wall for the purposes of eliciting a physiologic response, the method comprising:
providing a device comprising a base structure having a hydrophilic material presented therewith and an electrode structure thereon;
selectively positioning the device on a vessel wall and extending the base structure around at least a portion thereof; and
activating, deactivating, or otherwise modulating the device to provide stimulation to the vessel wall with the electrode for the purposes of eliciting a physiologic response.

28. The method of claim 27, wherein the step of providing stimulation to the vessel wall is for the purposes of eliciting a baroreflex.

29. The method of claim 27, further comprising determining an effective position for providing stimulation to the vessel wall before or during selectively positioning the device on the vessel wall.

30. The method of claim 29, wherein said step of determining an effective position comprises applying an electrical stimulus and observing a response.

31. The method of claim 27, wherein the vessel wall includes one or more baroreceptors therein, the step of selectively positioning the device on the vessel wall comprising determining the location of the one or more baroreceptors and effecting movement of the device such that the device is proximate the one or more baroreceptors.

32. The method of claim 27, further comprising providing electrical stimulation with the base structure, such that the stimulation is directionally provided toward the vessel wall.
